(19) United States
(12) Patent Application Publication
(10) Pub. No.: US 2026/0262641 A1
(43) Pub. Date: Sep. 10, 2026

(54) USE OF FENPYROXIMATE FOR CONTROLLING EARTH MITES, GROUND PEARLS AND ERIOPHYIDAES

(71) Applicant: ADAMA MAKHTESHIM LTD., Beer Sheva (IL)

(72) Inventor: Andrew HORSFIELD, Bracken Ridge, Queensland (AU)

(73) Assignee: ADAMA MAKHTESHIM LTD., Beer Sheva (IL)

(21) Appl. No.: 19/167,223

(22) PCT Filed: Mar. 21, 2024
(86) PCT No.: PCT/IL2024/050290

§ 371 Date: Sep. 19, 2025

Related U.S. Application Data
(60) Provisional application No. 63/491,407, filed on Mar. 21, 2023.

(51) Int. Cl. A01N 43/56; A01N 25/04; A01N 25/14; A01P 7/02

(52) U.S. Cl. CPC A01N 43/56; A01N 25/04; A01N 25/14; A01P 7/02

(57) ABSTRACT

The present invention relates to methods for controlling an earth mite, ground pearl, or Eriophyidae in turfgrass, cereals or grass pastures, in loci where control of said earth mite, ground pearl, or Eriophyidae is desired, by application of fenpyroximate.

### TECHNICAL FIELD

The present invention relates to methods for controlling an earth mite, ground pearl, or Eriophyidae in turfgrass, cereals or grass pastures, in loci where control of said earth mite, ground pearl, or Eriophyidae is desired, by application of fenpyroximate.

### BACKGROUND ART

### Redlegged Earth Mite (RLEM) Control in Arable Crops and Pastures

RLEM (Halotydeus destructor) is a Penthaleidae species of earth mite that damage crops and pastures, and it is considered one of the most destructive and economically significant establishment pests of crops and pastures in Australia, New Zealand and South Africa. The main methods currently available for controlling RLEM include: (i) seed treatments with neonicotinoids and fipronil to prevent damage to emerging seedlings; (ii) broadcast spraying of synthetic pyrethroids and organophosphates to the soil and emerging crops/pastures in effected fields; and (iii) broadcast spraying of diafenthiuron to emerged crops such as canola. Issues associated with these methods include inconsistent/short period of protection from seed treatments, and widespread resistance to synthetic pyrethroids and organophosphates insecticides. In addition, diafenthiuron, which is the main alternative, is highly expensive, and its short residual activity is limited to emerged crops. For all these reasons, there is an increased need to develop more sustainable control options and chemistries with novel mode of actions to control this pest.

Protection of Turfgrass with Fenpyroximate

Ground pearls. Ground pearls Margaroides spp are a significant pest of turfgrass. Damage from ground pearls includes weakened, thin and drought stresses plants, and some dead patches. Currently, there are limited options available to control ground pearls and no registered products in Australia.

Couch mite. Couch mite (Aceria cynodoniensis), belonging to the Eriophyidae family, is a significant mite pest species of warm season turfgrass. Feeding damage to turf stands causes formation of witches' broom symptoms and a decline in turf vigor and appearance. Several miticides are registered for couch mite, but the development of resistance is an ongoing problem.

### SUMMARY OF INVENTION

In one aspect, disclosed herein is a method of controlling an earth mite (a Penthaleidae), e.g., a Halotydeus sp. such as Halotydeus destructor (RLEM), or a Penthaleus sp., said method comprising applying to a locus where control of said earth mite is desired, e.g., a field of crop, an effective amount of fenpyroximate, optionally in combination with an insecticide synergist, a compound having an insecticidal activity, or a mixture thereof.

In another aspect, the present invention generally further relates to a method for controlling an arthropod pest of turf, cereals or grass pastures, more particularly an arthropod pest selected from a ground pearl (a Margarodidae sp.) and an Eriophyidae, said method comprising applying to a locus where control of said arthropod pest of turf, cereals or grass pastures is desired an effective amount of fenpyroximate, optionally in combination with a soil penetrant.

In one particular such aspect, disclosed herein is a method of controlling a ground pearl (a Margarodidae sp.), e.g., Eumargarodes laingi (pink ground pearl), said method comprising applying to a locus where control of said ground pearl is desired an effective amount of fenpyroximate, optionally in combination with a soil penetrant such as a soil wetting agent, e.g., Pluronic® PE 6200, or a soil adjuvant. In certain embodiments, the locus to which fenpyroximate is applied is a field of a turfgrass, e.g., a warm- or cold-season turfgrass, and said method is in fact aimed at reducing the density of said ground pearl density in said field of a turfgrass, to thereby improve the growth and/or color and/or quality of said turfgrass.

In another particular such aspect, disclosed herein is a method of controlling an Eriophyidae in turfgrass, cereals or grass pastures, e.g., a mite such as Aceria cynodoniensis (couch mite) or Aceria tosichella (wheat curl mite), said method comprising applying to a locus where control of said Eriophyidae is desired an effective amount of fenpyroximate. In certain embodiments, the locus to which fenpyroximate is applied is a field of a turfgrass, e.g., a warm- or cold-season turfgrass, and said method is in fact aimed at improving the growth and/or color and/or quality of said turfgrass. In other embodiments, said locus is a cereal field or a grass pasture, said Eriophyidae is wheat curl mite, and said method is, e.g., aimed at protecting said cereal field or grass pasture from a virus infection.

### DETAILED DESCRIPTION

### RLEM and Other Mite Pests in Arable Crops and Pastures

Prior to the development of resistant populations, effective products for RLEM control included synthetic pyrethroids (SP), organophosphates (OP) and mixtures thereof. The SP and OP products are broad-spectrum, with rapid contact activity on soil and plants, and can be applied pre and/or post-sowing and after seeding emergence. RLEM often attack germinating seedlings before they emerge, and control prior to seedling emergence is therefore critical. Diafenthiuron was recently introduced for RLEM control, but is a less effective/flexible option for RLEM management than SP and OP products due to its limited contact activity and requirement for a host plant to be present.

While mitochondrial complex I electron transport inhibitor (METI) miticides are known for control of a range of mite species from common mite pest families such as Tetranychidae, Tenuipalpidae, Tarsonemidae, and Eriophyidae, there was no indication that this mode of action is capable of controlling RLEM.

Example 1 herein demonstrates that while the METI miticide pyridaben is not efficacious against RLEM, fenpyroximate is surprisingly highly efficacious against this mite. The range of concentrations tested for fenpyroximate produced a clear dose-response, with mite mortality observed at concentrations greater than 50 mg active ingredient/liter (a.i./L); and 93.8 to 100% control of RLEM at concentrations of 300 and 500 g a.i./L, respectively. The range of concentrations tested for pyridaben did not produce a clear dose-response, with less than 50% mortality even at the highest rates. Example 4 shows that fenpyroximate is efficacious on both insecticide susceptible- and insecticide resistant populations of RLEM, and can therefore control known multiple insecticide resistant RLEM biotypes, including biotypes with reduced resistance to diafenthiuron. Example 5 further shows that fenpyroximate is effective on RLEM as both a pre- and post-crop emergent application

Protection of Turfgrass with Fenpyroximate

Ground pearls. As shown in Example 2, application of fenpyroximate to couch turfgrass was effective for controlling ground pearls. Following the application of fenpyroximate, the density of eggs and adults was significantly reduced, and controlling ground pearls significantly improved the Normalized Difference Vegetation Index (NDVI) of the turf, demonstrating the ability of fenpyroximate to improve turf quality. These findings are highly surprising, given that fenpyroximate has low water solubility and ground pearls are soil insects occurring below the soil surface. The use of a soil wetting agent such as Pluronic® PE 6200, or a soil adjuvant would have facilitated the movement of the fenpyroximate into the target zone of the pest following sufficient rainfall and irrigation. Example 6 demonstrates that fenpyroximate is efficacious against ground pearl, wherein the level of control provided on both eggs and adults is similar to those provided by both thiamethoxam and imidacloprid, resulting in a significant improvement in turf vigor.

Couch mite. Example 3 further shows that application of fenpyroximate to couch mite was surprisingly effective for controlling said mite, resulted in significantly reduced incidence of mite damage (witches' broom symptom), and was superior to the standard miticides that did not reduce the incidence of damage. As specifically shown, turf vigor and NDVI were significantly improved by fenpyroximate and clofentezine, but not by abamectin, demonstrating that fenpyroximate can protect and improve turf quality. Example 7 demonstrates that fenpyroximate is efficacious against couch mite, providing a level of control (in terms of both density of couch mite and incidence of couch mite damage) that is similar to that provided by the standard miticide, clofentezine+abamectin, and resulting in a significant improvement in turf quality.

In one aspect, the present invention thus relates to a method of controlling an earth mite (a Penthaleidae) comprising applying to a locus where control of said earth mite is desired an effective amount of fenpyroximate.

The term “earth mite” or “Penthaleidae” refers to a family of mites that are major winter pests of a variety of crops and pastures in southern Australia. This family belongs to the superfamily Eupodoidea, which is classified as Arachnida belonging to the subphylum Chelicerata and the phylum Arthropoda.

In some embodiments, the earth mite controlled by the method disclosed herein is a species of the genus Halotydeus, e.g., Halotydeus bakerae, Halotydeus castellus, Halotydeus destructor (redlegged earth mite, RLEM), and Halotydeus spectatus. In particular such embodiments, said earth mite is RLEM.

In other embodiments, the earth mite controlled by the method disclosed herein is a species of the genus Penthaleus, e.g., Penthaleus falcatus, Penthaleus major, Penthaleus minor, and Penthaleus tectus.

In further embodiments, the earth mite controlled by the method disclosed herein is a species of the genus Chromotydaeus.

The term “locus where control of said earth mite is desired” as used herein refers not only to areas where an earth mite such as RLEM may already be developed, but also to areas that have not yet been attacked by said winter pest, and to areas under cultivation. Locus includes the crop and propagation material of the crop (all the generative parts of the crop such as seeds and vegetative plant material such as cuttings and tubers, which can be used for the multiplication of the plant). Examples of propagation material of the crop include seeds, tubers, spores, corms, bulbs, rhizomes, sprouts basal shoots, stolons, buds and other parts of plants, including seedlings and young plants, which could be transplanted after germination or after emergence from soil. Locus also includes the area surrounding the crop and the growing media of the crop, such as soil and crop field.

In certain embodiments, the locus treated by the method disclosed herein, i.e., the locus where control of said earth mite according to any one of the embodiments above is desired, is a field of crop. The term “crop” (or “plant”) as used herein refers to whole plants, plant organs (e.g., leaves, stems, twigs, roots, trunks, limbs, shoots, fruits, etc.), plant cells, or plant seeds. Non-limiting examples of crops include canola, cereals such as wheat, barley and oats, pulses such as faba beans, field peas and vetch, lupins, sunflower, poppies, lucerne, pasture legumes, grasses, broadleaf weeds, and pastures.

In certain particular embodiments, the earth mite controlled by the method disclosed herein is RLEM, and the locus where control of said earth mite is desired is selected from canola, wheat, barley, oats, lupins, sunflower, faba beans, field peas, poppies, lucerne, vetch, pasture legumes, and grasses.

In other particular embodiments, the earth mite controlled by the method disclosed herein is a Penthaleus sp., and the locus where control of said earth mite is desired is selected from canola, broadleaf weeds, pastures, cereals, and pulses.

According to the method disclosed herein for controlling an earth mite, the fenpyroximate may be applied to said locus either post emergence of said crop or prior to emergence of said crop. In certain embodiments, the fenpyroximate is applied to said locus prior to emergence of said crop, either prior to sowing of said crop or after sowing of said crop.

The amount of fenpyroximate applied according to the method disclosed herein is effective for controlling said earth mite, and may depend on certain parameters such as the particular crop treated and sensitivity of said crop to said earth mite, as well as the timing of the application, i.e., post emergence of said crop, after sowing but prior to emergence of said crop, or prior to sowing of said crop. More specifically, said amount may be from about 10 g active ingredient/hectare (ai/ha) to about 4000 g ai/ha, e.g., from about 20 g ai/ha to about 3600 g ai/ha, from about 40 g ai/ha to about 3200 g ai/ha, from about 80 g ai/ha to about 2800 g ai/ha, from about 120 g ai/ha to about 2400 g ai/ha, from about 160 g ai/ha to about 2000 g ai/ha, or from about 200, 300 or 400 g ai/ha to about 1000 g ai/ha.

In certain embodiments, the method for controlling an earth mite, as disclosed in any one of the embodiments above, comprises applying of said fenpyroximate to said locus, in combination with an insecticide synergist, a compound having an insecticidal activity, or a mixture thereof.

The term “insecticide synergist” as used herein refers to a compound that greatly enhances the toxicity of an insecticide, although it is usually practically nontoxic, i.e., incapable of killing a target pest, per se. Examples of insecticide synergists include, without being limited to, piperonyl butoxide, N-octyl bicycloheptene dicarboximide, maleic acid esters such as diethyl maleate, trans-phenylbutenone, piprotal, propyl isome, sesamex, sesamolin, and a sulfoxide, as well as derivatives and analogues thereof.

The term “compound having an insecticidal activity” as used herein refers to any compound which is toxic to insects, i.e., capable of killing insects, e.g., by acting upon the insect's nervous system (e.g., cholinesterase inhibition), or acting as a growth regulator or endotoxin. Non-limiting examples of compounds having an insecticidal activity include oils such as a mineral oil, vegetable oil or an esterified form thereof, refined or unrefined botanical oil, and neem oil; a plant extract or at least one compound such as a small molecule or peptide isolated therefrom, e.g., Azadirachtin that is isolated from the neem tree (Azadirachta indica)), a pyrethrin (a pesticide naturally found in particular chrysanthemum flowers) such as Pyrethrin I, Cinerin I, Jasmolin I, Pyrethrin II, Cinerin II, and Jasmolin II, derived from Chrysanthemun cinerariifolium, or a Clitoria ternatea (a cyclotide-bearing plant) extract rich in small disulfide-rich peptides; or a fatty acid monoester such as an octanoic acid monoester, decanoic acid monoester, and dodecanoic acid-monoester.

In particular embodiments, the method for controlling an earth mite disclosed herein comprises application of said fenpyroximate in combination with an insecticide synergist, a compound having an insecticidal activity, or a mixture thereof, wherein the ratio between the amount of fenpyroximate and the amount of said insecticide synergist/compound having an insecticidal activity/mixture thereof applied is from about 100:1 to about 1:100, respectively, e.g., from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, or from about 10:1 to about 1:10.

In another aspect, the present invention relates to a method of controlling a ground pearl (Margarodidae sp.) comprising applying to a locus where control of said ground pearl is desired an effective amount of fenpyroximate, optionally in combination with a soil penetrant such as a soil wetting agent or a soil adjuvant.

The term “ground pearl” refers to a member of a family of scale insects (small insects of the order Hemiptera, suborder Sternorrhyncha) within the superfamily Coccoidea.

In certain embodiments, the ground pearl controlled by the method disclosed herein is Eumargarodes laingi (pink ground pearl), Margarodes prieskaensis, Margarodes vitis, and Margarodes verdendalensis. In particular such embodiments, said ground pearl is pink ground pearl.

In certain embodiments, the method of controlling a ground pearl as defined in any one of the embodiments above comprises applying to a locus where control of said ground pearl is desired an effective amount of fenpyroximate; and in other embodiments, said method comprises applying to said locus an effective amount of fenpyroximate in combination with a soil penetrant.

The term “soil penetrant” as used herein generally refers to a compound, e.g., an alcohol or a petroleum distillate such as a polyacrylamide, capable of increasing/improving water penetration into dry hydrophobic soils thereby regulating soil moisture and thus assisting to loosen up hard clay soils, hydrate sandy soil, and allow for water to better reach plant roots. Soil penetrants work by making water wetter, i.e., enabling water droplets spread more easily and penetrate soils more readily, and consequently helping water move through the surface layer and keep the surface dry. In certain embodiments, the soil penetrant applied in combination with said fenpyroximate is a soil wetting agent, i.e., a compound capable of lowering the surface tension of the water which improves infiltration into the soil. In other embodiments, the soil penetrant applied in combination with said fenpyroximate is a soil adjuvant, i.e., a compound capable, when added to an insecticide formulation or a tank mix, of facilitating the mixing, application, and/or effectiveness of said insecticide.

In particular embodiments, the soil penetrant applied in combination with said fenpyroximate is a soil wetting agent. Examples of soil wetting agents include, without limiting, Pluronic® PE 3100, Pluronic® PE 3500, Pluronic® PE 4300, Pluronic® PE 6100, Pluronic® PE 6120, Pluronic® PE 6200, Pluronic® PE 6400, Pluronic® PE 6800, Pluronic® PE 7400, Pluronic® PE 8100, Pluronic® PE 9200, Pluronic® PE 9400, Pluronic® PE 10100, Pluronic® PE 10300, Pluronic® PE 10400, Pluronic® PE 10500, and Pluronic® PE 10500 Solution, all low-foaming, nonionic surfactants. In a particular embodiment exemplified herein, the soil penetrant applied in combination with said fenpyroximate is Pluronic® PE 6200.

In certain embodiments, the method of controlling a ground pearl as defined in any one of the embodiments above comprises applying to a locus where control of said ground pearl is desired an effective amount of fenpyroximate in combination with a soil penetrant, e.g., a soil wetting agent such as each one of those defined above. According to the present invention, the weight ratio between said fenpyroximate and said soil penetrant in said combination may be from about 10:1 to about 1:250, respectively. In particular embodiments, the weight ratio between said fenpyroximate and said soil penetrant in said combination is from about 5:1 to about 1:125, from about 2:1 to about 1:60, from about 1:1 to about 1:30, from about 1:2 to about 1:20, from about 1:3 to about 1:10, or from about 1:4 to about 1:8, respectively.

In certain embodiments, the method of controlling a ground pearl as defined in any one of the embodiments above comprises applying to a locus where control of said ground pearl is desired an effective amount of fenpyroximate in combination with a soil penetrant, e.g., a soil wetting agent such as each one of those defined above, wherein the weight ratio between said fenpyroximate and said soil penetrant in said combination may be from about 10:1 to about 1:250, respectively. Such a combination may be formulated in any way suitable for application to said locus, e.g., as a suspension concentrate (SC), emulsifiable concentrate (EC), water-dispersible granule (WG or WDG), or soluble granules (SG).

The amount of fenpyroximate applied according to the method disclosed herein is effective for controlling said ground pearl, and may depend on certain parameters such as the particular turfgrass grown in the locus treated and the sensitivity of said turfgrass to said ground pearl, as well as on whether said fenpyroximate is applied alone or in combination with a soil penetrant. More specifically, said amount may be from about 10 g ai/ha to about 4000 g ai/ha, e.g., from about 20 g ai/ha to about 3600 g ai/ha, from about 40 g ai/ha to about 3200 g ai/ha, from about 80 g ai/ha to about 2800 g ai/ha, from about 120 g ai/ha to about 2400 g ai/ha, from about 160 g ai/ha to about 2000 g ai/ha, or from about 200, 300 or 400 g ai/ha to about 1000 g ai/ha.

The term “locus where control of said ground pearl is desired” as used herein refers to any locus affected by ground pearl as defined herein, but particularly to a field of a turfgrass. In certain embodiments, said turfgrass is a warm-season turfgrass such as Bermudagrass (Cynodon dactylon), Blue Couch (Digitaria didactyla), Dwarf hybrid green couch (Cynodon dactylon x C. transvaalensis), Broadleaf carpet grass (Axonopus compressus), Brunswick grass (Paspalum nicorae), Indian bluegrass (Bothriochloa pertusa), Kikuyu (Pennisetum clandestinum), Lax panic grass (Panicum laxum), Centipedegrass (Eremochloa ophiuroides), Seashore Paspalum (Paspalum vaginatum), Saltgrass (Distichlis spicata), Seashore paspalum (Paspalum vaginatum), American Buffalograss (Buchloe dactyloides), Bahiagrass (Paspalum notatum), St. Augustinegrass (Stenotaphrum secundatum), and Zoysiagrass (Zoysia matrella, Zoysia macrantha, Zoysia japonica and Zoysia japonica x Zoysia tenuifolia). In other embodiments, said turfgrass is a cold-season turfgrass such as Bluegrass hybrid (Poa arachnifera x Poa pratensis), Browntop bentgrass (Agrostis capillaris), Creeping bentgrass (Agrostis stolonifera), Fine fescue (Festuca rubra, Festuca rubra var. commutate, Festuca ovina var. duriuscula), Kentucky bluegrass (Poa pratensis), Perennial ryegrass (Lolium perenne), and Tall fescue (Festuca arundinacea). In particular embodiments, the method disclosed herein is for controlling a ground pearl in a field of a turfgrass where control of said ground pearl is desired, and said method is aimed at reducing the density of said ground pearl in said field of turfgrass, thereby improving the growth and/or color and/or quality of said turfgrass.

In one particular such aspect, the present invention thus relates to a method of controlling a ground pearl in a field of turfgrass, e.g., a warm-season turfgrass or cold-season turfgrass, in need, to thereby, e.g., reduce the density of said ground pearl in said field of turfgrass, and consequently improve the growth and/or color and/or quality of said turfgrass, said method comprising applying to said field of turfgrass an effective amount of fenpyroximate, optionally in combination with a soil penetrant such as a soil wetting agent or a soil adjuvant.

In a further aspect, the present invention relates to a method of controlling an Eriophyidae in turfgrass, cereals or grass pastures, comprising applying to a locus where control of said Eriophyidae is desired an effective amount of fenpyroximate.

The term “Eriophyidae” refers to a member of a family of more than 200 genera of mites, which live as plant parasites, commonly causing galls or other damage to the plant tissues and hence known as gall mites.

In certain embodiments, the Eriophyidae controlled by the method disclosed herein is a mite, i.e., a small arachnid (eight-legged arthropod). In particular embodiments, said mite is an Aceria such as Aceria cynodoniensis (couch mite), Aceria cynodonis, Aceria zoysiae, Aceria slykhuisi, and Aceria tosichella (wheat curl mite), an Abacarus such as grain rust mite (Abacarus hystrix), or an Aculodes such as Aculodes mckenziei. In more particular such embodiments, said mite is couch mite or wheat curl mite.

The amount of fenpyroximate applied according to the method disclosed herein is effective for controlling said Eriophyidae in turfgrass, cereals or grass pasture, and may depend on certain parameters such as the particular turfgrass, cereal or grass pasture grown in the locus treated and the sensitivity of said turfgrass, cereal or grass pasture to said Eriophyidae. More specifically, said amount may be from about 10 g ai/ha to about 4000 g ai/ha, e.g., from about 20 g ai/ha to about 3600 g ai/ha, from about 40 g ai/ha to about 3200 g ai/ha, from about 80 g ai/ha to about 2800 g ai/ha, from about 120 g ai/ha to about 2400 g ai/ha, from about 160 g ai/ha to about 2000 g ai/ha, or from about 200, 300 or 400 g ai/ha to about 1000 g ai/ha.

The term “locus where control of said Eriophyidae is desired” as used herein refers to any locus affected by an Eriophyidae as defined herein, but particularly to a field of a turfgrass, cereal field, or grass pasture.

In certain embodiments, the locus where control of said Eriophyidae is desired is a field of turfgrass, and said turfgrass is a warm-season turfgrass such as Bermudagrass (Cynodon dactylon), Blue Couch (Digitaria didactyla), Dwarf hybrid green couch (Cynodon dactylon x C. transvaalensis), Broadleaf carpet grass (Axonopus compressus), Brunswick grass (Paspalum nicorae), Indian bluegrass (Bothriochloa pertusa), Kikuyu (Pennisetum clandestinum), Lax panic grass (Panicum laxum), Centipedegrass (Eremochloa ophiuroides), Seashore Paspalum (Paspalum vaginatum), Saltgrass (Distichlis spicata), Seashore paspalum (Paspalum vaginatum), American Buffalograss (Buchloe dactyloides), Bahiagrass (Paspalum notatum), St. Augustinegrass (Stenotaphrum secundatum), and Zoysiagrass (Zoysia matrella, Zoysia macrantha, Zoysia japonica and Zoysia japonica x Zoysia tenuifolia). In other embodiments, said turfgrass is a cold-season turfgrass such as Bluegrass hybrid (Poa arachnifera x Poa pratensis), Browntop bentgrass (Agrostis capillaris), Creeping bentgrass (Agrostis stolonifera), Fine fescue (Festuca rubra, Festuca rubra var. commutate, Festuca ovina var. duriuscula), Kentucky bluegrass (Poa pratensis), Perennial ryegrass (Lolium perenne), and Tall fescue (Festuca arundinacea). In particular such embodiments, the method disclosed herein is for controlling an Eriophyidae in a field of a turfgrass where control of said Eriophyidae is desired, and said method is aimed at improving the growth and/or color and/or quality of said turfgrass.

In other embodiments, the locus where control of said Eriophyidae is desired is a cereal field, and said cereal is, e.g., wheat, barley, oat, millet, cereal rye, or triticale. In particular such embodiments, said Eriophyidae is wheat curl mite, known as a vector for spreading and transmission of viruses like wheat streak mosaic virus (WMSV) and wheat mosaic virus (WMoV), and the method disclosed is for, e.g., protecting the cereal grown in said field from a virus infection, e.g., WMSV or WMoV infection.

In further embodiments, the locus where control of said Eriophyidae is desired is a grass pasture such as ryegrass or cocksfoot. In particular such embodiments, said Eriophyidae is wheat curl mite, and the method disclosed is for, e.g., protecting the grass pasture in said field from a virus infection, e.g., WMSV or WMoV infection.

In one particular such aspect, the present invention thus relates to a method of controlling an Eriophyidae, e.g., couch mite, in a field of turfgrass, e.g., a warm-season turfgrass or cold-season turfgrass, in need, e.g., to thereby improve the growth and/or color and/or quality of said turfgrass, said method comprising applying to said field of turfgrass an effective amount of fenpyroximate.

In another particular such aspect, the present invention thus relates to a method of controlling an Eriophyidae, e.g., wheat curl mite, in a cereal field in need, e.g., to thereby protect the cereal grown in said field from a virus, e.g., WMSV or WMoV, infection, said method comprising applying to said cereal field an effective amount of fenpyroximate.

In a further particular such aspect, the present invention thus relates to a method of controlling an Eriophyidae, e.g., wheat curl mite, in a grass pasture in need, e.g., to thereby protect said grass pasture from a virus, e.g., WMSV or WMoV, infection, said method comprising applying to said grass pasture an effective amount of fenpyroximate.

Based on the present disclosure, which provides methods for controlling both a ground pearl (a Margarodidae sp.) and Eriophyidae, the present invention may generally be referred to as relating, inter alia, to a method for controlling an arthropod pest of turf, cereals or grass pasture, more particularly selected from a ground pearl and an Eriophyidae such as a mite, said method comprising applying to a locus where control of said arthropod pest of turf, cereals or grass pasture is desired an effective amount of fenpyroximate, optionally in combination with a soil penetrant.

In certain embodiments, the method for controlling an arthropod pest of turf, cereals or grass pasture disclosed herein is aimed at controlling a ground pearl such as pink ground pearl, Margarodes prieskaensis, Margarodes vitis, and Margarodes verdendalensis, but particularly pink ground pearl, and comprises applying to a locus where control of said ground pearl is desired, e.g., a field of a turfgrass, an effective amount of fenpyroximate, optionally in combination with a soil penetrant such as a soil wetting agent or a soil adjuvant, according to any one of the embodiments above.

In other embodiments, the method for controlling an arthropod pest of turf, cereals or grass pasture disclosed herein is aimed at controlling an Eriophyidae, e.g., a mite such as an Aceria including couch mite, Aceria cynodonis, Aceria zoysiae, Aceria slykhuisi, and wheat curl mite, an Abacarus such as grain rust mite (Abacarus hystrix), or an Aculodes such as Aculodes mckenziei, but particularly couch mite or wheat curl mite, and comprises applying to a locus where control of said Eriophyidae is desired, e.g., a cereal field or grass pasture, an effective amount of fenpyroximate, according to any one of the embodiments above.

The term “locus where control of said arthropod pest of turf, cereals or grass pasture is desired” as used herein refers to any locus affected by an arthropod pest of turf, cereals, or grass pasture, more particularly a ground pearl (a Margarodidae sp.) or an Eriophyidae, each as defined above, but particularly to a field of a turfgrass which may be, e.g., a warm-season turfgrass or a cold-season turfgrass, a cereal field, or a grass pasture.

Unless otherwise indicated, all numbers expressing, e.g., amounts of components or ratios between components, used in this specification, are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting Examples.

### EXAMPLES

### Example 1. Fenpyroximate is Effective in Controlling Halotydeus Destructor

### Methods

Laboratory bioassays were undertaken to determine the efficacy of the mitochondrial complex I electron transport inhibitor (METI) miticides fenpyroximate and pyridaben against redlegged earth mite (H. destructor; RLEM), following the glass vial technique developed by Hoffmann et al. (1997), but adapted for plastic vials (Arthur et al., 2021). On the 14^{th} of May 2022, H. destructor were collected from capeweed (Arctotheca calendula) in South Morang, Victoria (−37.625, 145.110), a site with no known history of insecticide application. Mites were collected via suction using a Stihl SH55 blower vacuum, placed in plastic containers with paper towel and vegetation, and transported back to the laboratory, where they were stored at 4° C. until testing.

Serial dilutions were prepared for each insecticide (Table 1), with the addition of a non-ionic surfactant (Tween 20) at 0.01% to aid the spread of the insecticide and ensure even coating. This concentration of Tween 20 has been previously shown to have no toxic effect on H. destructor. Ten concentrations of fenpyroximate ranging from the proposed field rate of 2000 mL diluted in 100 L of water (1000 mg a.i./L) to 1×10⁻⁵ were serially diluted and tested along with a water control. Nine concentrations of pyridaben ranging from the proposed field rate of 1480 mL diluted in 100 L of water (1998 mg a.i./L) to 1×10⁻⁵ were serially diluted and tested along with a water control. Eight concentrations of omethoate, ranging from 10 times the field rate of 100 mL diluted in 100 L of water (290 mg a.i./L) to 1×10⁻⁵, were tested along with a water control. Seven concentrations of bifenthrin, ranging from 10 times the field rate of 40 mL diluted in 100 L of water (100 mg a.i./L) to 1×10⁻⁵, were tested along with a water control. For each insecticide concentration tested, approximately 10 mL of solution was poured into a 15 mL plastic vial and swirled to ensure a complete coating, with excess liquid removed. Six vials were coated per concentration and were left to dry overnight. Control vials were treated in the same way, except water (with the addition of 0.01% Tween) was used.

Approximately eight individual H. destructor were then placed into each vial along with 2-3 leaves of common vetch (Vicia sativa), which provided food and humidity. Vials were then sealed with a screw lid and placed in a controlled temperature cabinet at 18° C. After 24-hours exposure, mites were scored as alive (moving freely), incapacitated (inhibited movement), or dead (no movement over a 5 second period). Incapacitated individuals were pooled with dead individuals for analysis, as they invariably die and therefore do not contribute to the next generation (Umina and Hoffman, 1999).

TABLE 1
Insecticides used in this study

| Insecticide | Active ingredient | Concentration | |
|---|---|---|---|
| Fenpyroximate SC | Fenpyroximate | 50 | g/L |
| Pyridaben EC | Pyridaben | 135 | g/L |
| Le-Mat | Omethoate | 290 | g/L |
| Astral 250EC | Bifenthrin | 250 | g/L |

### Data Analysis

Dose-response curves were generated by plotting percentage mortality against log concentration. Concentrations that caused 50% mortality (lethal concentration, LC₅₀) (along with 95% confidence intervals, CIs) were estimated from observations of mortality using a binomial logistic regression (Robertson and Preisler 1992; Venables and Ripley 2002). Logistic regression is well suited for the analysis of binary response data (e.g., dead/alive). All analyses were conducted using R version 3.3.1 (R Development Core Team 2022).

### Results

Control mortality was 0% across all bioassays conducted. Mortality responses of H. destructor to concentrations of fenpyroximate SC and pyridaben indicate that fenpyroximate SC is toxic to H. destructor, but not pyridaben. The range of concentrations tested for fenpyroximate SC (0.01-1000 mg a.i./L) produced a clear dose-response with mortality responses from 0% to 100%. However, mortality only occurred between 50 mg a.i./L-1000 mg a.i./L, with zero mortality below 50 mg a.i./L. The range of concentrations tested for pyridaben (0.01998-1998 mg a.i./L), did not produced a clear dose-response, with 45% mortality being the highest mortality observed. The commercial standards, omethoate and bifenthrin, produced clear dose response curves with mortality responses from 0% to 100% at the concentrations tested (Table 2).

The LC₅₀ value for fenpyroximate SC was estimated to be 128.97 mg a.i./L, which was much higher than the LC₅₀ values for the commercial standards omethoate (0.681 mg a.i./L) and bifenthrin (0.085 mg a.i./L) (Table 3). An accurate LC₅₀ value was not able to be calculated for pyridaben, given the low levels of mortality observed.

TABLE 2
RLEM mortality after 24 hours exposure
to varying insecticides and rates

| Treatment | Rate (mg a.i./L) | % RLEM mortality |
|---|---|---|
| Untreated | — | 0 |
| Omethoate | 0.003 | 0.0 |
| | 0.03 | 2.8 |
| | 0.3 | 11.7 |
| | 0.9 | 55.3 |
| | 2.9 | 98.1 |
| | 29 | 100 |
| | 290 | 100 |
| | 2900 | 100 |
| Bifenthrin | 0.001 | 0.0 |
| | 0.01 | 0.0 |
| | 0.1 | 64.9 |
| | 0.3 | 98.7 |
| | 1 | 100 |
| | 10 | 100 |
| | 100 | 100 |
| Fenpyroximate 50 SC | 0.01 | 0.0 |
| | 0.1 | 0.0 |
| | 1 | 0.0 |
| | 10 | 0.0 |
| | 30 | 0.0 |
| | 50 | 6.0 |
| | 100 | 30.5 |
| | 300 | 93.8 |
| | 500 | 100 |
| | 1000 | 98.7 |
| Pyridaben 135 EC | 0.02 | 0.0 |
| | 0.2 | 0.0 |
| | 2.0 | 0.0 |
| | 20 | 0.0 |
| | 60 | 0.0 |
| | 200 | 9.0 |
| | 599 | 23.4 |
| | 999 | 45.5 |
| | 1998 | 27.5 |

### Conclusions

This study demonstrates that fenpyroximate is efficacious against RLEM, but pyridaben is not. The range of concentrations tested for fenpyroximate produced a clear dose-response, with mite mortality observed at concentrations greater than 50 mg a.i./L; and 93.8 to 100% control of RLEM at concentrations of 300 and 500 g a.i./L, respectively. The range of concentrations tested for pyridaben did not produce a clear dose-response, with less than 50% mortality even at the highest rates.

TABLE 3
LC₅₀ values (95% CI) and regression coefficients (±se)
for H. destructor computed from binomial logistic
models for responses to fenpyroximate SC, pyridaben,
omethoate and bifenthrin after 24-hour exposure

| | Regression slope | | |
|---|---|---|---|
| Chemical | coefficients (±se) | LC₅₀ (95% CI) (mg a.i./L) | |
| Fenpyroximate SC | 3.194 (±0.418) | 128.97 | (108.969-152.637) |
| Pyridaben | 0.966 (±0.173) | NA | |
| Omethoate | 1.940 (±0.328) | 0.681 | (0.526-0.881) |
| Bifenthrin | 3.519 (±1.153) | 0.085 | (0.067-0.107) |

### Example 2. Fenpyroximate is Effective in Controlling Ground Pearl

### Methods

A field trial was conducted in kikuyu turf, to evaluate fenpyroximate for control of ground pearl (Margarodes spp). The trial was located at Sydney University, New South Wales (−33.888, 151.188), in an area with a known infestation of ground pearl.

On the 9th and 30th of November 2021 and 21^{st} of December 2021, fenpyroximate 50 SC was applied at 200 g ai/ha, together with PE 6200 soil wetting agent, and compared to Meridian and Kohinor 350, both applied at the same timings and with the same soil wetting agent. All treatments were applied using a hand boom fitted with XR11004 nozzles, in a spray volume of 430 L/ha.

The ground pearl density by species was assessed by sampling 20 cores per plot and recording the number of eggs and adults, and Normalized Difference Vegetation Index (NDVI) was assessed 56 days after the third application using a Greenseeker.

### Data Analysis

All data was statistically analyzed using analysis of variance and Fisher's protected least significant different test (Fishers LSD). The level of significance for all tests was P<0.05.

### Results

The density of ground pearl eggs and adults was significantly reduced by fenpyroximate and thiamethoxam as shown in Table 4. Imidacloprid did not reduce ground pearl egg density and was significantly less effective than fenpyroximate. All insecticides significantly reduced the density of adult ground pearl.

Fenpyroximate significantly improved turf color and quality by maintaining the NDVI, while the untreated plots declined in both color and vigor (Table 5). The effect on NVDI was similar to thiamethoxam and imidacloprid.

TABLE 4
Efficacy of fenpyroximate on ground pearl

| | | | | Ground pearls/plot | | | |
|---|---|---|---|---|---|---|---|
| | | Active ingredient | Product rate | Eggs | | Adults | |
| No. | Product | and concentration | (mL/ha) | Pre-spray | 56 DAA3 | Pre-spray | 56 DAA3 |
| 1 | Untreated | — | — | 14.8 a | 17.0 d | 10.0 a | 25.5 b |
| 2 | Meridian | Thiamethoxam | 1200 | 7.8 a | 8.5 abc | 15.5 a | 9.8 a |
| | PE 6200 | Soil adjuvant | 25000 | | | | |
| 3 | Kohinor 350 SC | Imidacloprid 350 SC | 1500 | 9.0 a | 12.5 cd | 10.5 a | 13.8 a |
| | PE 6200 | Soil wetting agent | 25000 | | | | |
| 4 | Fenpyroximate | Fenpyroximate 50 SC | 4000 | 10.3 a | 5.3 a | 4.0 a | 12.8 a |
| | PE 6200 | Soil wetting agent | 25000 | | | | |

Means followed by the same letter do not significantly differ (P < 0.05)
DAA = days after application

TABLE 5
The effect of fenpyroximate on NDVI, 56 days after the third application

| | | Active ingredient | Product rate | NDVI | | | |
|---|---|---|---|---|---|---|---|
| No. | Product | and concentration | (mL/ha) | Pre-spray | | 56 DAA3 | |
| 1 | Untreated | — | — | 7.8 | a | 6.9 | b |
| 2 | Meridian | Thiamethoxam | 1200 | 7.5 | a | 7.5 | a |
| | PE 6200 | Soil wetting agent | 25000 | | | | |
| 3 | Kohinor 350 SC | Imidacloprid 350 SC | 1500 | 8.25 | a | 7.8 | a |
| | PE 6200 | Soil wetting agent | 25000 | | | | |
| 4 | Fenpyroximate | Fenpyroximate 50 SC | 4000 | 8.0 | a | 7.6 | a |

| | | |
|---|---|---|
| PE 6200 | Soil adjuvant | 25000 |

Means followed by the same letter do not significantly differ (P < 0.05)

### Conclusions

This study demonstrates that fenpyroximate is efficacious against ground pearl. The level of control provided by fenpyroximate was similar to thiamethoxam on both eggs and adults. The level of control provided by fenpyroximate was superior to imidacloprid on eggs and similar on adults. There was a significant and equivalent improvement in NDVI from fenpyroximate, thiamethoxam and imidacloprid.

### Example 3. Fenpyroximate is Effective in Controlling Couch Mite

A field trial was conducted in Santa Ana couch turfgrass, to evaluate fenpyroximate for control of couch mite (Aceria cynodoniensis). The trial was located at Terrey Hills Golf and Country Club, New South Wales (−33.668, 151.217). On the 8th and 11th November 2021, fenpyroximate 50 SC was applied at 200 g ai/ha and compared to Prophesy and Thumper (Table 6). All treatments were applied using a hand boom fitted with XR11004 nozzles and in a spray volume of 430 L/ha.

The incidence of couch mite damage, to the terminal growth point (known as witches' broom) was assessed 42 days after the second application. Turf biomass and vigor were assessed at the same timing on a 0 to 10 rating scale, along with NDVI using a Greenseeker.

### Data Analysis

All data was statistically analyzed using analysis of variance and Fisher's protected least significant different test (Fishers LSD). The level of significance for all tests was P<0.05.

### Results

The incidence of couch mite damage was significantly reduced by fenpyroximate as shown in Table 6. Neither of the standard miticides were effective at reducing the incidence of damage.

Fenpyroximate significantly improved turf biomass and vigor and NDVI as shown in Tables 7-8. Clofentezine also improved turf biomass and vigor and NDVI, but not abamectin.

### Conclusions

This study demonstrates that fenpyroximate is efficacious against couch mite. The level of control provided by fenpyroximate was superior to the standard miticides, clofentezine and abamectin. The vigor, biomass and NDVI responses to fenpyroximate, were similar to clofentezine and superior to abamectin.

TABLE 6
Efficacy of fenpyroximate on couch mite

| | | Active ingredient | Product rate | % Incidence mite damage | | |
|---|---|---|---|---|---|---|
| No. | Product | and concentration | (mL/ha) | Pre-spray | 42 DAA2 | |
| 1 | Untreated | — | — | 93.3 a | 65.3 | d |
| 2 | Prophesy | Clofentezine 500 SC | 500 | 94.0 a | 52.0 | bcd |
| 3 | Thumper | Abamectin 20 ME | 2000 | 95.3 a | 57.0 | cd |
| | BS 1000 | Non-ionic surfactant | 0.25% v/v | | | |
| 4 | Fenpyroximate | Fenpyroximate 50 SC | 4000 | 95.8 a | 47.0 | abc |

Means followed by the same letter do not significantly differ (P < 0.05)
DAA = days after application

TABLE 7
Effect of fenpyroximate on turf vigor, 42 days after the third application

| | | Active ingredient | Product rate | Biomass and vigor (0-10) | | |
|---|---|---|---|---|---|---|
| No. | Product | and concentration | (mL/ha) | Pre-spray | 42 DAA2 | |
| 1 | Untreated | — | — | 3.6 a | 4.0 | c |
| 2 | Prophesy | Clofentezine 500 SC | 500 | 4.0 a | 4.9 | a |
| 3 | Thumper | Abamectin 20 ME | 2000 | 3.9 a | 4.4 | abc |
| | BS 1000 | Non-ionic surfactant | 0.25% v/v | | | |
| 4 | Fenpyroximate | Fenpyroximate 50 SC | 4000 | 4.1 a | 4.8 | ab |

Means followed by the same letter do not significantly differ (P < 0.05)
DAA = days after application

TABLE 8
Effect of fenpyroximate on NDVI, 42 days after the third application

| | | Active ingredient | Product rate | NDVI | | |
|---|---|---|---|---|---|---|
| No. | Product | and concentration | (mL/ha) | Pre-spray | 42 DAA2 | |
| 1 | Untreated | — | — | 0.50 a | 0.54 | d |
| 2 | Prophesy | Clofentezine 500 SC | 500 | 0.53 a | 0.64 | a |
| 3 | Thumper | Abamectin 20 ME | 2000 | 0.52 a | 0.58 | a-d |
| | BS 1000 | Non-ionic surfactant | 0.25% v/v | | | |
| 4 | Fenpyroximate | Fenpyroximate 50 SC | 4000 | 0.55 a | 0.63 | ab |

Means followed by the same letter do not significantly differ (P < 0.05)
DAA = days after application

### Example 4. Fenpyroximate is Effective in Controlling Resistant Biotypes of Halotydeus Destructor

### Methods

Laboratory bioassays were undertaken to determine the efficacy of the fenpyroximate and diafenthiuron against an insecticide-susceptible and insecticide-resistant population of redlegged earth mite (H. destructor; RLEM), following the glass vial technique developed by Hoffmann et al. (1997), but adapted for plastic vials (Arthur et al., 2021). The insecticide-susceptible H. destructor population tested in this bioassay was collected from burr medic (Medicago polymorpha) and barley grass (Hordeum leporinum) in Moulamein, NSW (−35.102, 144.026), a site with no known history of insecticide application. The insecticide-resistant H. destructor population was collected from a pasture paddock in Tooperany, SA, where a moderate to high level of resistance to synthetic pyrethroids and low-level resistance to organophosphates was identified during resistance surveillance in 2022. Mites were collected via suction using a Stihl SH55 blower vacuum, placed in plastic containers with paper towel and vegetation, and transported back to the laboratory, where they were stored at 4° C. until testing.

Serial dilutions were prepared for each insecticide (Table 9), with the addition of a non-ionic surfactant (Tween 20) at 0.01% to aid the spread of the insecticide and ensure even coating. Seven concentrations of fenpyroximate were tested, ranging from 1×10⁻⁴ to 10 times the proposed field rate of 2000 mL diluted in 100 L of water (1000 mg a.i./L). Nine concentrations of bifenthrin were tested, ranging from 1×10⁻⁵ to 10 times the field rate of 40 mL diluted in 100 L of water (100 mg a.i./L). Seven concentrations of omethoate were tested, ranging from 1×10⁻⁵ to 10 times the field rate of 100 mL in 100 L of water (290 mg a.i./L).

Eight concentrations of diafenthiuron were tested, ranging from 1×10⁻⁵ to 30 times the field rate of 600 mL diluted in 100 L of water (3000 mg a.i./L).

Six vials were coated per concentration and were left to dry overnight. Control vials were treated in the same way, except water (with the addition of 0.01% Tween) was used. The next day, eight individual H. destructor were then placed into each vial along with 2-3 leaves of common vetch (Vicia sativa), which provided food and humidity. Vials were then sealed with a screw lid and placed in a controlled temperature cabinet at 18° C. After 24-hours exposure, mites were scored as alive (moving freely), incapacitated (inhibited movement), or dead (no movement over a 5 second period). Incapacitated individuals were pooled with dead individuals for analysis, as they invariably die and therefore do not contribute to the next generation (Umina and Hoffman, 1999).

TABLE 9
Insecticides used in this study

| Insecticide | Active ingredient | Concentration | |
|---|---|---|---|
| Fenpyroximate SC | Fenpyroximate | 50 | g/L |
| Receptor | Diafenthiuron | 500 | g/L |
| Talstar 250 EC | Bifenthrin | 250 | g/L |
| Le Mat | Omethoate | 290 | g/L |

### Data Analysis

Dose-response curves were generated by plotting percentage mortality against log concentration. A series of logistic models were used to quantify the sensitivity of H. destructor to each chemical and to test whether any differences in sensitivity were present between the two populations. Logistic regression is well suited for the analysis of binary response data (e.g., dead/alive). Each model included chemical concentration and H. destructor population as fixed-effect predictors, which allowed testing for whether populations had average differences in mortality (i.e., model intercept) via F tests. It was then determined if populations had differences in mortality that were dependent on chemical concentration (i.e., differences in regression slopes) by comparing the inclusion of a population×concentration predictor (i.e., allowing slopes to vary between populations). The concentrations causing 50% mortality (lethal concentration, LC₅₀ values) were then estimated, along with 95% confidence intervals (CIs) from the full model (additive+interactive predictors). The resulting LC₅₀ values were used to quantify the level of insecticide resistance (resistance ratios) relative to the known insecticide-susceptible population. All analyses were conducted using R version 4.3.1 (R Development Core Team 2023).

### Results

Control mortality was below 9% across the bioassays conducted (Table 10). There were no significant differences between the insecticide-susceptible and resistant populations in response to fenpyroximate indicating both populations exhibited similar levels of sensitivity (Table 11). LC₅₀ values were estimated to be 39.189 (21.04-72.99) mg a.i./L and 51.54 (28.41-93.52) mg a.i./L for the susceptible and resistant populations, respectively. Additionally, no differences in regression slopes between the susceptible and resistant population was observed (Table 11).

There were significant population differences between the insecticide-susceptible and resistant populations in response to diafenthiuron, with the resistant population exhibiting a significantly higher LC₅₀ value than the susceptible population (Table 11). LC₅₀ values were estimated to be 48.35 (21.87-106.91) mg a.i./L and 244.01 (111.76-532.78) mg a.i./L for the susceptible and resistant populations, respectively (Table 11). No differences in regression slopes between the susceptible and resistant population was observed (Table 11).

A significant difference between the resistant and susceptible population in response to bifenthrin was present (Table 11). LC₅₀ values were estimated to be 0.053 (0.036-0.081) mg a.i./L for the susceptible population and 14.05 (2.35-84.09) mg a.i./L for the resistant population, confirming a moderate to high level of resistance to pyrethroids. Regression slopes also differed between the susceptible and resistant populations (Table 11).

For omethoate, the dose-response curve of the resistant population was shifted to the right with respect to the susceptible population, but no statistically significant difference between these populations was present (Table 11). LC₅₀ values were estimated to be 0.348 (0.182-0.664) mg a.i./L for the susceptible population and 0.935 (0.387-2.258) mg a.i./L for the resistant population. Additionally, no difference in regression slopes between the susceptible and resistant population was observed (Table 11).

TABLE 10
RLEM mortality after 24 hours exposure
to varying insecticides and rates

| | | % RLEM mortality | |
|---|---|---|---|
| Treatment | Rate (mg a.i./L) | Susceptible | Resistant |
| Fenpyroximate 50 SC | 0 | 8.9 | 1.4 |
| | 0.1 | 6.9 | 4.6 |
| | 1 | 12.6 | 12.9 |
| | 10 | 11.3 | 11.2 |
| | 100 | 47.3 | 45.7 |
| | 300 | 92.2 | 86.3 |
| | 1000 | 98.5 | 100.0 |
| | 10000 | 100.0 | 100.0 |
| Diafenthiuron | 0 | 8.9 | 1.4 |
| | 0.03 | 13.9 | 3.9 |
| | 0.3 | 15.6 | 10.6 |
| | 3 | 13.0 | 13.2 |
| | 30 | 21.5 | 15.7 |
| | 300 | 64.1 | 39.5 |
| | 3000 | 100.0 | 75.5 |
| | 30000 | 100.0 | 100.0 |
| | 90000 | 100.0 | 100.0 |
| Bifenthrin | 0 | 8.9 | 1.4 |
| | 0.001 | 7.0 | 4.4 |
| | 0.01 | 4.7 | 9.3 |
| | 0.03 | 17.4 | 5.8 |
| | 0.1 | 74.3 | 22.3 |
| | 0.3 | 98.1 | 44.6 |
| | 1 | 100.0 | 48.9 |
| | 10 | 100.0 | 70.9 |
| | 100 | 100.0 | 59.0 |
| | 1000 | 100.0 | 53.5 |
| Omethoate | 0 | 8.9 | 1.4 |
| | 0.0029 | 7.6 | 16.1 |
| | 0.029 | 10.1 | 18.9 |
| | 0.29 | 32.1 | 17.2 |
| | 2.9 | 95.8 | 47.5 |
| | 29 | 100.0 | 100.0 |
| | 290 | 100.0 | 100.0 |
| | 2900 | 100.0 | 100.0 |

### Conclusions

This study demonstrates that fenpyroximate is efficacious on both a known insecticide susceptible population of RLEM and an insecticide resistant population. The resistant RLEM population was significantly less susceptible to bifenthrin and diafenthiuron, and had previously been observed to have low level resistance to omethoate. The key finding is that fenpyroximate can control known multiple insecticide resistant RLEM biotypes, including biotypes with reduced resistance to diafenthiuron.

TABLE 11
LC₅₀ values (95% CI) and regression coefficients (±se) for insecticide susceptible
and resistant H. destructor populations computed from binomial logistic models
for responses to fenpyroximate SC, diafenthiuron, bifenthrin and omethoate after
24-hour exposure. Different superscript lowercase letters indicate significant
population differences within each chemical bioassay at the P < 0.05 level.

| | | Regression slope | LC₅₀ (95% CI) | | |
|---|---|---|---|---|---|
| Chemical | Population | coefficients (±se) | (mg a.i./L) | | Resistance ratio* |
| Fenpyroximate SC | Susceptible | 0.748 (±0.107) | 39.189 | a | — |
| | Resistant | 0.777 (±0.115) | 51.544 | a | — |
| Diafenthiuron | Susceptible | 0.488 (±0.059) | 48.353 | a | — |
| | Resistant | 0.480 (±0.059) | 244.010 | b | 5.04 |
| Bifenthrin | Susceptible | 1.482 (±0.282) | 0.053 | a | — |
| | Resistant | 0.232 (±0.047) | 14.050 | b | 265.09 |
| Omethoate | Susceptible | 0.899 (±0.154) | 0.348 | a | — |
| | Resistant | 0.514 (±0.076) | 0.935 | a | — |

*Calculated by dividing the LC₅₀ for resistant insects by the LC₅₀ for susceptible insects.

### Example 5. Fenpyroximate is Effective in Controlling Halotydeus destructor as a Pre- or Post-Emergent Application

A field trial was conducted in canola cv. Garrison, to evaluate fenpyroximate for control of redlegged earth mite (H. destructor; RLEM) as a pre- or post-emergent application. The trial was located at Beverley, Western Australia (−32.2248085, 117.0268493). The canola was sown on 13th June 2023. Fenpyroximate 50 SC was applied at 50 and 100 g ai/ha on 29th June 2023 to bare soil prior to crop emergence, or on 20th July 2023 at the cotyledon to two leaf stage. All treatments were applied using a hand boom fitted with flat fan nozzles in a spray volume of 100 L/ha.

The density of RLEM was assessed 33 days after the second application timing by counting the number of mites per plant.

### Data Analysis

All data was statistically analyzed using analysis of variance and Fisher's protected least significant different test (Fishers LSD). The level of significance for all tests was P<0.05.

### Results

The density of RLEM was significantly reduced by fenpyroximate at both rates and application timings (Table 12). The level of control was comparable to the standards at both timings.

TABLE 12
Efficacy of fenpyroximate on redlegged earth mite in canola

| | | Active ingredient | Product rate | Application | % Control | |
|---|---|---|---|---|---|---|
| No. | Product | and concentration | (mL/ha) | timing | (33 DAA2) | |
| 1 | Untreated | — | — | — | 0 | d |
| 2 | Pyrinex Super | Bifenthrin 20 + | 500 | A | 75 | a |
| | | Chlorpyrifos 400 EC | | | | |
| 3 | Fenpyroximate 50 SC | Fenpyroximate 50 SC | 1000 | A | 67 | abc |
| 4 | Fenpyroximate 50 SC | Fenpyroximate 50 SC | 2000 | A | 61 | abc |
| 5 | Receptor | Diafenthiuron 500 SC | 400 | B | 54 | bc |
| 6 | Le-mat | Omethoate 290 SL | 100 | B | 74 | a |
| 7 | Fenpyroximate 50 SC | Fenpyroximate 50 SC | 1000 | B | 63 | abc |
| 8 | Fenpyroximate 50 SC | Fenpyroximate 50 SC | 2000 | B | 68 | ab |

Means followed by the same letter do not significantly differ (P < 0.05)
A—post-sow pre-emergent
B—early post-emergent (1 to 2 leaf canola)
DAA = days after application

### Conclusions

This study demonstrates that fenpyroximate is efficacious against RLEM at both rates evaluated. Fenpyroximate is effective on RLEM as both a pre and post emergent application.

### Example 6. Fenpyroximate is Effective in Controlling Ground Pearl

### Methods

A field trial was conducted in kikuyu turf, to evaluate fenpyroximate for control of ground pearl (Margarodes spp). The trial was located at Robina, Queensland (−28.069442, 153.401817), in an area with a known infestation of ground pearl.

On the 29th of November 2022, 20th of December 2022 and 10th of January 2023, fenpyroximate 50 SC was applied at 200 g ai/ha, together with Stamina Balance soil wetting agent, and compared to Meridian and Kohinor 350, both applied at the same timings and with the same soil wetting agent. All treatments were applied using a hand boom fitted with XC08 nozzles, in a spray volume of 443 L/ha.

The ground pearl density by species was assessed prior to the first application and again 21 days after the third application by sampling 20 cores per plot and recording the number of eggs and adults. Turf vigor was assessed on a 0 to 10 rating scale (0=dead; 10=best quality/color) prior to the first application and 42 days after the third application.

### Data Analysis

All data was statistically analyzed using analysis of variance and Fisher's protected least significant different test (Fishers LSD). The level of significance for all tests was P<0.05.

### Results

The density of ground pearl eggs and adults was significantly reduced by fenpyroximate, thiamethoxam and imidacloprid as shown in Table 13. The level of control was similar for all three insecticides.

Fenpyroximate, thiamethoxam and imidacloprid significantly improved turf vigor, while the untreated plots declined in both color and vigor (Table 14). The effect of fenpyroximate on turf vigor was similar to thiamethoxam and imidacloprid.

TABLE 13
Efficacy of fenpyroximate on ground pearl

| | | | | Ground pearls/plot | | | |
|---|---|---|---|---|---|---|---|
| | | Active ingredient | Product rate | Eggs | | Adults | |
| No. | Product | and concentration | (mL or g/ha) | Pre-spray | 21 DAA3 | Pre-spray | 21 DAA3 |
| 1 | Untreated | — | — | 4.7 | 3.0 de | 9.3 | 8.3 d |
| 2 | Meridian | Thiamethoxam | 1200 | 3.0 | 0.7 abc | 6.0 | 1.3 ab |
| | Stamina B | Soil adjuvant | 20000 | | | | |
| 3 | Kohinor 350 SC | Imidacloprid 350 SC | 1500 | 2.0 | 0.7 abc | 5.7 | 1.3 ab |
| | Stamina B | Soil wetting agent | 20000 | | | | |
| 4 | Fenpyroximate | Fenpyroximate 50 SC | 4000 | 4.0 | 1.0 abc | 6.7 | 1.0 ab |
| | Stamina B | Soil wetting agent | 20000 | | | | |

Means followed by the same letter do not significantly differ (P < 0.05)
DAA = days after application

### Conclusions

This study demonstrates that fenpyroximate is efficacious against ground pearl. The level of control provided by fenpyroximate was similar to both thiamethoxam and imidacloprid on both eggs and adults. There was a significant and equivalent improvement in turf vigor from fenpyroximate, thiamethoxam and imidacloprid.

TABLE 14
The effect of fenpyroximate on turf vigor, 42 days after the third application

| | | Active ingredient | Product rate | Turf vigor (0-10) | |
|---|---|---|---|---|---|
| No. | Product | and concentration | (mL/ha) | Pre-spray | 56 DAA3 |
| 1 | Untreated | — | — | 5.0 | 5.2 b |
| 2 | Meridian | Thiamethoxam | 1200 | 5.1 | 6.0 a |
| | Stamina B | Soil adjuvant | 20000 | | |
| 3 | Kohinor 350 SC | Imidacloprid 350 SC | 1500 | 5.1 | 5.9 a |
| | Stamina B | Soil wetting agent | 20000 | | |
| 4 | Fenpyroximate | Fenpyroximate 50 SC | 4000 | 5.1 | 6.2 a |
| | Stamina B | Soil wetting agent | 20000 | | |

Means followed by the same letter do not significantly differ (P < 0.05)

### Example 7. Fenpyroximate is Effective in Controlling Couch Mite

A field trial was conducted in hybrid couch turfgrass, to evaluate fenpyroximate for control of couch mite (Aceria cynodoniensis). The trial was located at Oxley Golf Club, Oxley Queensland (−27.57077, 152.99762). On 2^{nd} and 16^{th} August 2023, fenpyroximate 50 SC was applied at 100 g ai/ha and compared to Abazine Miticide (Table 15). All treatments were applied using a hand boom fitted with XR11004 nozzles and in a spray volume of 430 L/ha.

The severity of couch mite damage to the terminal growth point (known as witches' broom) was assessed pre-spraying, then at 42 and 56 days after the second application. The density of couch mite was assessed pre-spraying and at 28 days after the second application. Turf quality was assessed on a 0 to 9 rating scale (0=dead; 9=best quality/color) prior to the first application and 14 days after the second application.

### Data Analysis

All data was statistically analyzed using analysis of variance and Fisher's protected least significant different test (Fishers LSD). The level of significance for all tests was P<0.05.

### Results

The incidence of couch mite damage was significantly reduced by fenpyroximate as shown in Table 15. Fenpyroximate provided a similar reduction in damage to the standard miticide, Abazine. In addition, and similar to Abazine, fenpyroximate significantly both reduced the density of couch mite as shown in Table 16, and improved turf quality as shown in Table 17.

TABLE 15
Efficacy of fenpyroximate on couch mite

| | | Active ingredient | Product rate | % Severity mite damage | | |
|---|---|---|---|---|---|---|
| No. | Product | and concentration | (mL/ha) | Pre-spray | 42 DAA2 | 56 DAA2 |
| 1 | Untreated | — | — | 47.5 | 58.0 b | 64.0 c |
| 2 | Abazine | Abamectin 18 + | 1350 | 40.5 | 14.0 a | 16.5 ab |
| | Miticide | Clofentezine 187.5 SC | | | | |
| 3 | Fenpyroximate | Fenpyroximate 50 SC | 2000 | 40.0 | 7.0 a | 6.5 a |

Means followed by the same letter do not significantly differ (P < 0.05)
DAA = days after application

TABLE 16
Effect of fenpyroximate on couch mite density, 28 days after the third application

| | | Active ingredient | Product rate | Couch mites/3 terminals | | |
|---|---|---|---|---|---|---|
| No. | Product | and concentration | (mL/ha) | Pre-spray | 28 DAA2 | |
| 1 | Untreated | — | — | 127.3 | 135.3 | b |
| 2 | Abazine | Abamectin 18 + | 1350 | 63.5 | 26.8 | a |
| | Miticide | Clofentezine 187.5 SC | | | | |
| 3 | Fenpyroximate | Fenpyroximate 50 SC | 2000 | 130.5 | 45.8 | a |

Means followed by the same letter do not significantly differ (P < 0.05)
DAA = days after application

TABLE 17
Effect of fenpyroximate on turf quality, 14 days after the second application

| | | Active ingredient | Product rate | Turf quality (0-9) | | |
|---|---|---|---|---|---|---|
| No. | Product | and concentration | (mL/ha) | Pre-spray | 14 DAA2 | |
| 1 | Untreated | — | — | 3.8 | 3.3 | c |
| 2 | Abazine | Abamectin 18 + | 1350 | 4.3 | 5.3 | ab |
| | Miticide | Clofentezine 187.5 SC | | | | |
| 3 | Fenpyroximate | Fenpyroximate 50 SC | 2000 | 4.5 | 5.1 | ab |

Means followed by the same letter do not significantly differ (P < 0.05)
DAA = days after application

### Conclusions

This study demonstrates that fenpyroximate is efficacious against couch mite. The level of control provided by fenpyroximate was similar to the standard miticide, clofentezine+abamectin. The turf quality improvement from fenpyroximate was also similar to clofentezine+abamectin.

### REFERENCES

Arthur A L, Maino J, Hoffmann A A, Jasper M, Lord A, Micic S, Edwards O, van Rooyen A, Umina P A. Learnings from over a decade of increasing pesticide resistance in the redlegged earth mite, Halotydeus destructor (Tucker). Pest Management Science, 2021, 77, 3013-3024
Hoffmann A A, Porter S, Kovacs I. 1997. The response of the major crop and pasture pest, the redlegged earth mite (Halotydeus destructor) to pesticides: dose-response curves and evidence of tolerance. Experimental and Applied Acarology, 1997, 21, 151-162
R Core Team. 2022. R: A language and environment for statistical computing. R Foundation for Statistical Computing, Vienna, Austria. http://www.R-project.org/.
Robertson J L, Preisler H K. 1992. Bioassays with Arthropods. CRC: Boca Raton.
Umina P A. Pyrethroid resistance discovered in a major agricultural pest in southern Australia: the redlegged earth mite Halotydeus destructor (Acari: Penthaleidae). Pest Management Science, 2007, 63, 1185-1190
Umina P A, Hoffmann A A. Tolerance of cryptic species of blue oat mites (Penthaleus spp.) and the redlegged earth mite (Halotydeus destructor) to pesticides. Australian Journal of Experimental Agriculture, 1999, 39, 621-628
Venables W N, Ripley B D. 2002. Modern Applied Statistics with S. Springer: New York. ISBN 0-387-95457-0, 2002. http://www.stats.ox.ac.uk/pub/MASS4

1. A method of controlling an earth mite (Penthaleidae) comprising applying to a locus where control of said earth mite is desired an effective amount of fenpyroximate.

2. The method of claim 1, wherein said earth mite is a Halotydeus sp.

3. The method of claim 2, wherein said Halotydeus sp. is selected from Halotydeus bakerae, Halotydeus castellus, Halotydeus destructor (Redlegged earth mite, RLEM), and Halotydeus spectatus.

4. The method of claim 3, wherein said Halotydeus sp. is RLEM.

5. The method of claim 1, wherein said earth mite is a Penthaleus sp.

6. The method of claim 5, wherein said Penthaleus sp. is selected from Penthaleus falcatus, Penthaleus major, Penthaleus minor, and Penthaleus tectus.

7. The method of claim 1, wherein said earth mite is a Chromotydaeus sp.

8. The method of any one of claims 1-7, wherein said locus is a field of crop.

9. The method of claim 8, wherein said crop is selected from canola, wheat, barley, oats, lupins, sunflower, faba beans, field peas, poppies, lucerne, vetch, pasture legumes, grasses, broadleaf weeds, pastures, cereals, and pulses.

10. The method of claim 8, wherein said earth mite is RLEM, and said crop is selected from canola, wheat, barley, oats, lupins, sunflower, faba beans, field peas, poppies, lucerne, vetch, pasture legumes, and grasses.

11. The method of claim 8, wherein said earth mite is a Penthaleus sp. and said crop is canola, broadleaf weeds, pastures, cereals, and pulses.

12. The method of any one of claims 8-11, wherein said fenpyroximate is applied to said locus post emergence of said crop.

13. The method of any one of claims 8-11, wherein said fenpyroximate is applied to said locus prior to emergence of said crop, either prior to or after sowing of said crop.

14. The method of any one of claims 1-13, wherein said fenpyroximate is applied to said locus in combination with an insecticide synergist, a compound having an insecticidal activity, or a mixture thereof.

15. The method of claim 14, wherein said insecticide synergist is selected from piperonyl butoxide, N-octyl bicycloheptene dicarboximide, piprotal, propyl isome, sesamex, sesamolin, a sulfoxide, and a derivative or analogue thereof; or said compound having an insecticidal activity is an oil such as a mineral oil, vegetable oil or an esterified form thereof, refined or unrefined botanical oil, and neem oil; a plant extract or at least one compound such as a small molecule or peptide isolated therefrom; or a fatty acid monoester such as an octanoic acid-, decanoic acid-, and dodecanoic acid-monoester.

16. A method for controlling an arthropod pest of turf, cereals or grass pastures, selected from a ground pearl (a Margarodidae sp.) and an Eriophyidae, said method comprising applying to a locus where control of said arthropod pest of turf, cereals or grass pastures is desired an effective amount of fenpyroximate, optionally in combination with a soil penetrant.

17. The method of claim 16, for controlling a ground pearl (Margarodidae sp.), said method comprising applying to a locus where control of said ground pearl is desired an effective amount of fenpyroximate, optionally in combination with a soil penetrant such as a soil wetting agent or a soil adjuvant.

18. The method of claim 17, wherein said Margarodidae sp. is selected from Eumargarodes laingi (pink ground pearl), Margarodes prieskaensis, Margarodes vitis, and Margarodes verdendalensis.

19. The method of claim 18, wherein said Margarodidae sp. is pink ground pearl.

20. The method of any one of claims 17-19, wherein said soil penetrant is a soil wetting agent.

21. The method of claim 20, wherein said soil wetting agent is selected from Pluronic® PE 3100, Pluronic® PE 3500, Pluronic® PE 4300, Pluronic® PE 6100, Pluronic® PE 6120, Pluronic® PE 6200, Pluronic® PE 6400, Pluronic® PE 6800, Pluronic® PE 7400, Pluronic® PE 8100, Pluronic® PE 9200, Pluronic® PE 9400, Pluronic® PE 10100, Pluronic® PE 10300, Pluronic® PE 10400, Pluronic® PE 10500, and Pluronic® PE 10500 Solution.

22. The method of claim 21, wherein said soil wetting agent is Pluronic® PE 6200.

23. The method of any one of claims 17-22, wherein the weight ratio between said fenpyroximate and said soil penetrant in said combination is from about 10:1 to about 1:250, respectively.

24. The method of claim 23, wherein the weight ratio between said fenpyroximate and said soil penetrant in said combination is from about 5:1 to about 1:125, from about 2:1 to about 1:60, from about 1:1 to about 1:30, from about 1:2 to about 1:20, from about 1:3 to about 1:10, or from about 1:4 to about 1:8, respectively.

25. The method of any one of claims 17-24, wherein said fenpyroximate and said soil penetrant are formulated as a suspension concentrate (SC), emulsifiable concentrate (EC), water-dispersible granule (WG or WDG), or soluble granules (SG).

26. The method of any one of claims 17-25, wherein said locus is a field of a turfgrass.

27. The method of claim 26, wherein said turfgrass is a warm-season turfgrass such as Bermudagrass (Cynodon dactylon), Blue Couch (Digitaria didactyla), Dwarf hybrid green couch (Cynodon dactylon x C. transvaalensis), Broadleaf carpet grass (Axonopus compressus), Brunswick grass (Paspalum nicorae), Indian bluegrass (Bothriochloa pertusa), Kikuyu (Pennisetum clandestinum), Lax panic grass (Panicum laxum), Centipedegrass (Eremochloa ophiuroides), Seashore Paspalum (Paspalum vaginatum), Saltgrass (Distichlis spicata), Seashore paspalum (Paspalum vaginatum), American Buffalograss (Buchloe dactyloides), Bahiagrass (Paspalum notatum), St. Augustinegrass (Stenotaphrum secundatum), and Zoysiagrass (Zoysia matrella, Zoysia macrantha, Zoysia japonica and Zoysia japonica x Zoysia tenuifolia); or a cold-season turfgrass such as Bluegrass hybrid (Poa arachnifera x Poa pratensis), Browntop bentgrass (Agrostis capillaris), Creeping bentgrass (Agrostis stolonifera), Fine fescue (Festuca rubra, Festuca rubra var. commutate, Festuca ovina var. duriuscula), Kentucky bluegrass (Poa pratensis), Perennial ryegrass (Lolium perenne), and Tall fescue (Festuca arundinacea).

28. The method of claim 26 or 27, for reducing the density of said ground pearl in said field of a turfgrass, thereby improving the growth and/or color and/or quality of said turfgrass.

29. The method of claim 16, for controlling an Eriophyidae, said method comprising applying to a locus where control of said Eriophyidae is desired an effective amount of fenpyroximate.

30. The method of claim 29, wherein said Eriophyidae is a mite.

31. The method of claim 30, wherein said mite is an Aceria such as Aceria cynodoniensis (couch mite), Aceria cynodonis, Aceria zoysiae, Aceria slykhuisi, and Aceria tosichella (wheat curl mite), an Abacarus such as grain rust mite (Abacarus hystrix), or an Aculodes such as Aculodes mckenziei.

32. The method of any one of claims 29-31, wherein said locus is a field of a turfgrass.

33. The method of claim 32, wherein said turfgrass is a warm-season turfgrass such as Bermudagrass (Cynodon dactylon), Blue Couch (Digitaria didactyla), Dwarf hybrid green couch (Cynodon dactylon x C. transvaalensis), Broadleaf carpet grass (Axonopus compressus), Brunswick grass (Paspalum nicorae), Indian bluegrass (Bothriochloa pertusa), Kikuyu (Pennisetum clandestinum), Lax panic grass (Panicum laxum), Centipedegrass (Eremochloa ophiuroides), Seashore Paspalum (Paspalum vaginatum), Saltgrass (Distichlis spicata), Seashore paspalum (Paspalum vaginatum), American Buffalograss (Buchloe dactyloides), Bahiagrass (Paspalum notatum), St. Augustinegrass (Stenotaphrum secundatum), and Zoysiagrass (Zoysia matrella, Zoysia macrantha, Zoysia japonica and Zoysia japonica x Zoysia tenuifolia); or a cold-season turfgrass such as Bluegrass hybrid (Poa arachnifera x Poa pratensis), Browntop bentgrass (Agrostis capillaris), Creeping bentgrass (Agrostis stolonifera), Fine fescue (Festuca rubra, Festuca rubra var. commutate, Festuca ovina var. duriuscula), Kentucky bluegrass (Poa pratensis), Perennial ryegrass (Lolium perenne), and Tall fescue (Festuca arundinacea).

34. The method of claim 32 or 33, for improving the growth and/or color and/or quality of said turfgrass.

35. The method of any one of claims 29-31, wherein said locus is a cereal field or grass pasture.

36. The method of claim 35, wherein said Eriophyidae is Aceria tosichella (wheat curl mite), and said method is for protecting said cereal field or grass pasture from a virus infection.
